(19) United States
(12) Patent Application Publication
(10) Pub. No.: US 2011/0313252 A1
(43) Pub. Date: Dec. 22, 2011

(54) MICRO SENSING APPARATUS

(75) Inventor: WEI-TENG LIN, NEW TAIPEI CITY (TW)

(73) Assignee: PIONEER MEDICAL INSTRUMENT CO., LTD., NEW TAIPEI CITY (TW)

(21) Appl. No.: 13/118,667

(22) Filed: May 31, 2011

(30) Foreign Application Priority Data
Jun. 18, 2010 (TW) 099120021

(51) Int. Cl. A61B 1/06; H05K 7/00

(52) U.S. Cl. 600/162; 361/679.01

(57) ABSTRACT

The present invention discloses a micro sensing apparatus comprising a sensing device, a cylindrical plastic body and a plurality of circuits. The sensing device has a bottom surface. The cylindrical plastic body is axially coupled to the sensing device and includes a connecting portion and a carrying portion, and the connecting portion has a first end surface coupled to the bottom surface, and the carrying portion is integrally formed and coupled to the connecting portion, and the carrying portion includes a plurality of electronic devices installed thereon and a plurality of circuits formed on the carrying portion. With the integrally formed connecting portion and carrying portion, the problem of requiring an adapting mechanism of a conventional sensing device to couple a printed circuit board can be solved. The cylindrical plastic body is axially coupled to the sensing device to reduce the overall external diameter of the micro sensing apparatus.

### CROSS-REFERENCE TO RELATED APPLICATIONS

This non-provisional application claims priority under 35 U.S.C. §119(a) on patent application No(s). 099120021 filed in Taiwan, R.O.C. on Jun. 18, 2010, the entire contents of which are hereby incorporated by reference.

### BACKGROUND OF THE INVENTION

1. Field of the Invention

The present invention relates to a micro sensing apparatus, in particular to the area of integrally forming an adapting mechanism of a micro sensing device coupled to a circuit structure of an electronic device.

2. Description of the Related Art

In recent years, various electronic devices such as mobile phones, camcorders, cameras and endoscopic devices usually have a sensing device for providing a sensing function, and these electronic devices with a photographic function generally require a smaller sensing device for carrying out the aforementioned photographic function.

A conventional micro sensing device generally adopts an adapting mechanism and couples a rigid printed circuit board and a sensing device to both corresponding ends of the adapting mechanism by soldering. However, incomplete solders or poor contacts may occur during the soldering process and result in an unstable transmission of electric signals. If the micro sensing device adopts a flexible printed circuit board coupled onto the adapting mechanism by an adhesion, the flexible printed circuit board may fall off and fail to transmit electric signals if it is not adhered properly.

In FIG. 1, if the sensing device 11 is installed onto the printed circuit board 0 directly, the area of upper and lower surfaces of the printed circuit board 0 can be used for installing a very limited quantity of electronic devices 18. If the number of electronic devices 18 required on the printed circuit board 0 increases, the external diameter of the printed circuit board 0 will be increased, thus the overall external diameter of the micro sensing device 1 will increase accordingly, such that the external diameter of the micro sensing device 1 cannot fit the external diameter of the micro sensing device 11.

In view of the aforementioned shortcomings of the prior art, the inventor of the present invention discloses a micro sensing device to overcome the shortcomings of the prior art.

### SUMMARY OF THE INVENTION

Therefore, it is a primary objective of the present invention to provide a micro sensing apparatus that adopts integrally formed connecting portion and carrying portion to overcome the problem of requiring an adapting mechanism to couple a sensing device to a printed circuit and also solve the problems of having a too-large external diameter of the sensing device that may not fit the external diameter of the micro sensing apparatus.

To achieve the foregoing objective, the present invention provides a micro sensing apparatus comprising a sensing device, a cylindrical plastic body and a plurality of circuits. The sensing device has a bottom surface. The cylindrical plastic body is axially coupled to the sensing device, and includes a connecting portion and a carrying portion, wherein the connecting portion has a first end surface coupled to the bottom surface, and the carrying portion is integrally formed and coupled to the connecting portion, and the carrying portion includes a plurality of electronic devices installed thereon. The circuits are formed at the carrying portion. With the integrally formed connecting portion and carrying portion, the problem of requiring the use of an adapting mechanism in a conventional sensing device to couple a printed circuit board can be solved.

The micro sensing apparatus of the present invention has the following advantages:

(1) The sensing device can be installed to the integrally formed connecting portion and carrying portion to simplify the installation procedure and improve the convenience.

(2) The sensing device can be installed to the integrally formed connecting portion and carrying portion to overcome the problems caused by soldering, adhering or snapping the printed circuit board onto the adapting mechanism, and prevent the printed circuit board from falling off due to external vibration forces.

(3) An easy transmission of electric signals can be achieved by using circuits (such as the curved surface circuit, the three-dimensional circuit or a combination of the curved circuit and the planar circuit) formed on a surface of the cylindrical plastic body.

(4) A large quantity of the electronic devices can be installed on the surface of the surface of the cylindrical plastic body, so that the cylindrical plastic body of the invention can prevent a too-large external diameter of the micro sensing apparatus that may not fit the external diameter of the miniaturized sensing device.

(5) The cylindrical plastic body can be axially coupled to the sensing device to reduce the overall external diameter of the micro sensing apparatus significantly.

The detailed structure, operating principle and effects of the present invention will now be described in more details hereinafter with reference to the accompanying drawings that show various embodiments of the invention as follows.

### BRIEF DESCRIPTION OF THE DRAWINGS

FIG. 1 is a schematic perspective view of a conventional micro sensing device;

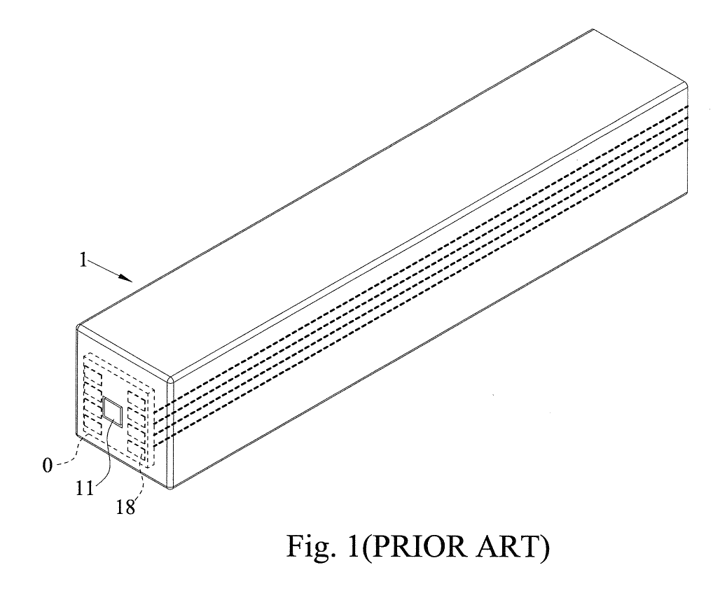

FIG. 2A is a schematic view of assembling a micro sensing apparatus in accordance with a first preferred embodiment of the present invention;

FIG. 2B is a perspective view of a micro sensing apparatus in accordance with a first preferred embodiment of the present invention;

FIG. 3 is a cross-sectional view of a micro sensing apparatus in accordance with a second preferred embodiment of the present invention used as an endoscopic device and an endoscope;

FIG. 4 is a perspective view of a micro sensing apparatus in accordance with a third preferred embodiment of the present invention;

FIG. 5 is a perspective view of a micro apparatus device in accordance with a fourth preferred embodiment of the present invention;

FIG. 6 is a perspective view of a micro sensing apparatus in accordance with a fifth preferred embodiment of the present invention;

FIG. 7 is a perspective view of a micro sensing apparatus in accordance with a sixth preferred embodiment of the present invention; and

FIG. 8 is a perspective view of a micro sensing apparatus in accordance with a seventh preferred embodiment of the present invention.

### DETAILED DESCRIPTION OF THE PREFERRED EMBODIMENTS

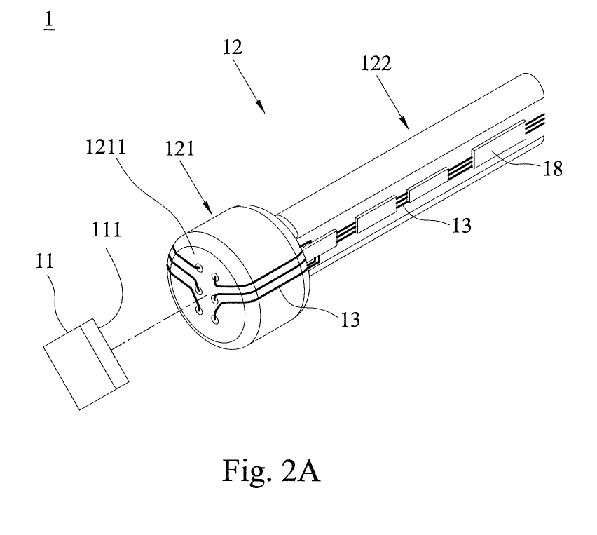

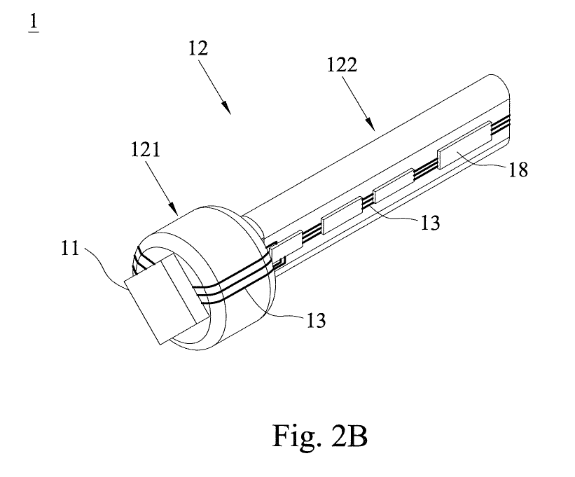

With reference to FIGS. 2A and 2B for a schematic view and a perspective view of a micro sensing apparatus in accordance with a first preferred embodiment of the present invention respectively, the micro sensing apparatus 1 has an external diameter from 1 mm to 15 mm, and the micro sensing apparatus 1 comprises a sensing device 11, a cylindrical plastic body 12 and a plurality of circuits 13.

The sensing device 11 has a bottom surface 111, wherein the sensing device 11 can be a complementary metal oxide semiconductor (CMOS) image sensing device, a charge coupled device (CCD) image sensing device, a thermal sensing device, a pressure sensing device, a flow sensing device, a laser sensing device, a displacement sensing device, an ultrasonic sensing device or an infrared sensing device, but the present invention is not limited to the aforementioned sensing devices only. The sensing device 11 is provided for sensing an electronic device such as a related semiconductor component including a mini printed circuit board, a chip or a transistor, and the electronic device is not shown in the figure and will not be described in details here.

The cylindrical plastic body 12 is axially coupled to the sensing device 11 to achieve the effect of reducing the overall external diameter of the micro sensing apparatus 1 significantly. The cylindrical plastic body 12 can be in a circular cylindrical shape, a rectangular cylindrical shape, a triangular cylindrical shape, an elliptical cylindrical shape, a semi-circular cylindrical shape, or a hyperbolic shape. The cylindrical plastic body 12 includes a connecting portion 121 and a carrying portion 122, wherein the connecting portion 121 has a first end surface 1211 coupled to the bottom surface 111 of the sensing device 111. In this preferred embodiment, the bottom surface 111 of the sensing device 11 is soldered onto the first end surface 1211 of the connecting portion 121, but the invention is not limited to such arrangement only. The first end surface 1211 can be a planar surface, and the first end surface 1211 can also be a circular surface, a rectangular surface, a triangular surface, an elliptical surface, a semicircular surface or a curved surface.

The first end surface 1211 of the cylindrical plastic body 12 can be coupled to the sensing device 11 by a surface mount technology (SMT). In SMT, a layer of soldering material (such as tin, aluminum, copper or nickel paste) is coated onto contact points of the first end surface 1211, and then the sensing device 11 is put into a high-temperature soldering oven and heated to melt the soldering material, such that the sensing device 11 can be adhered and coupled closely onto the first end surface 1211 of the cylindrical plastic body 12.

The cylindrical plastic body 12 can be formed by an injection molding method, a hollow air-blow molding method, a vacuum molding method, or a hot press molding method. In this preferred embodiment, the injection molding method is used as an example for illustrating the present invention, but the invention is not limited to such arrangement only. In the injection molding method, particulate plastic materials are poured into a high-temperature storage cylinder through a funnel, such that the particulate plastic material becomes a liquid plastic, an the liquid plastic is pushed by a hydraulic pressure to inject the melted liquid plastic into a low-temperature die mold for a cooling process and condense the liquid plastic to form a 3D product. The cylindrical plastic body 12 can reduce the weight of the conventional metal parts, such that if more electronic devices 18 are installed on the surface of the cylindrical plastic body 12 (since the conventional printed circuit board can have a very small quantity of electronic devices installed on both upper and lower surfaces of the printed circuit board), the external diameter of the micro sensing apparatus 1 still can fit the small external diameter of the sensing device 11.

The carrying portion 122 is integrally formed and coupled to the connecting portion 121, and the carrying portion 122 is provided for installing a plurality of electronic devices 18. Wherein, the carrying portion 122 can be in shape of a board or a column, such as a circular cylindrical shape, a rectangular cylindrical shape, a triangular cylindrical shape, an elliptical cylindrical shape, a semi-circular cylindrical shape, or a hyperbolic cylindrical shape, such that a side (or an end) of the carrying portion 122 can be integrally formed with a side (or an end) of the connecting portion 121.

The plurality of circuits 13 can be formed on a surface of the carrying portion 122 by a coating, laminating, printing or electroplating method. The circuits 13 can be made of tin foil, aluminum foil, nickel foil or copper foil, and one of the circuits 13 includes a curved-surface circuit, a three-dimensional circuit, or a combination of a curved-surface circuit and a planar circuit to achieve the effect of connecting the image sensor 11 and the electronic device 18 easily for transmitting electric signals. However, the present invention is not limited to such arrangement only.

In this preferred embodiment, the circuits 13 can be formed inside the connecting portion 121 (as indicated by the dotted line of the circuit 13 in FIG. 2A) or on a surface of the connecting portion 121 (as indicated by the solid line of the circuit 13 in FIG. 2A). This circuit 13 is in contact with the bottom surface 111 of the sensing device 11 and extended to the surface of the carrying portion 122 through the interior of the connecting portion 121 or the surface of the connecting portion 121.

The micro sensing apparatus 1 can be manufactured by a molded interconnect device (MID) process or a laser direct structuring (LDS) process. In this preferred embodiment, the LDS process is used for illustrating the invention, but the invention is not limited to this method only. The micro sensing apparatus 1 is formed by the LDS and the plastic material is processed by a plastic injection molding process to produce the cylindrical plastic body 12, and then the laser is applied to the cylindrical plastic body 12 for a surface treatment and an activation treatment, such that the circuits 13 can be adhered with the cylindrical plastic body 12. And then, the conductive metal is electroplated on the manufactured surface. Finally, the electronic device 18 is put into a high-temperature soldering oven and soldered onto the carrying portion 122 of the cylindrical plastic body 12, and the sensing device 11 in the high-temperature soldering oven is soldered onto the first end surface 1211 of the connecting portion 121 of the cylindrical plastic body 12.

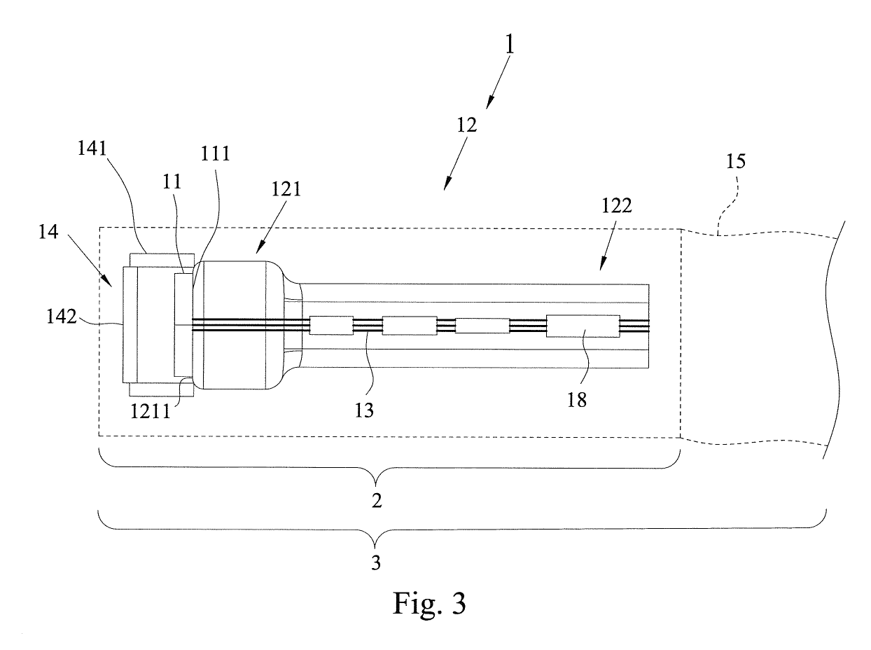

With reference to FIG. 3 for a cross-sectional view of a micro sensing apparatus in accordance with a second preferred embodiment of the present invention, the functions and components of the micro sensing apparatus of this preferred embodiment are substantially the same as those of the first preferred embodiment, and thus will not be described here again, and the only difference shown in the figure resides on that if a lens module 14 is installed at the front end of the micro sensing apparatus 1, an endoscopic device 2 will be formed; and if a lens module 14 is installed at the front end of the micro sensing apparatus 1 and a hollow pipe 15 is installed at a rear end of the micro sensing apparatus 1 or a light emitting element (not shown in the figure) is installed for providing illumination, an endoscope 3 will be formed. The lens module 14 is aligned towards the sensing device 11 and includes a casing 141 and at least one lens 142, wherein the lens 142 is disposed in the casing 141, and the hollow pipe 15 is in proximity to the carrying portion 122.

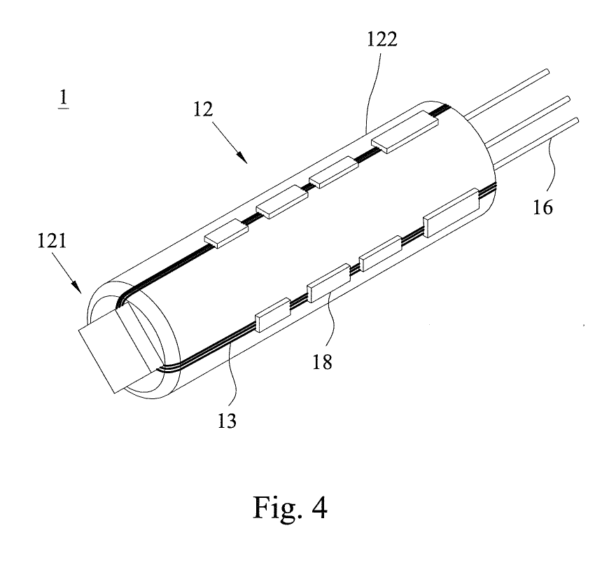

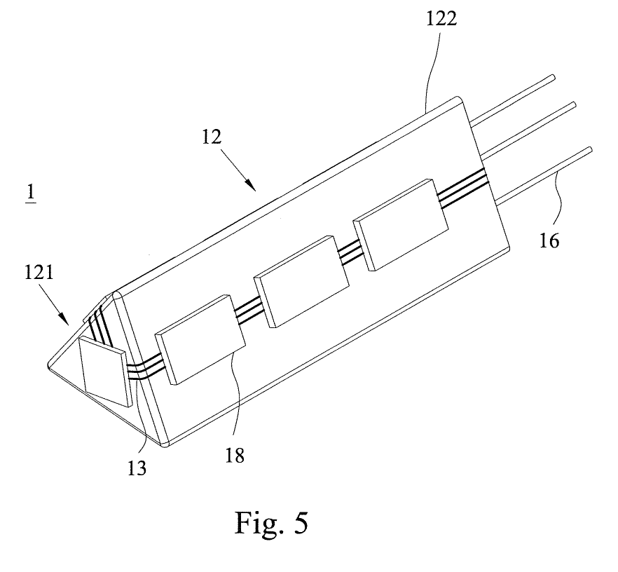

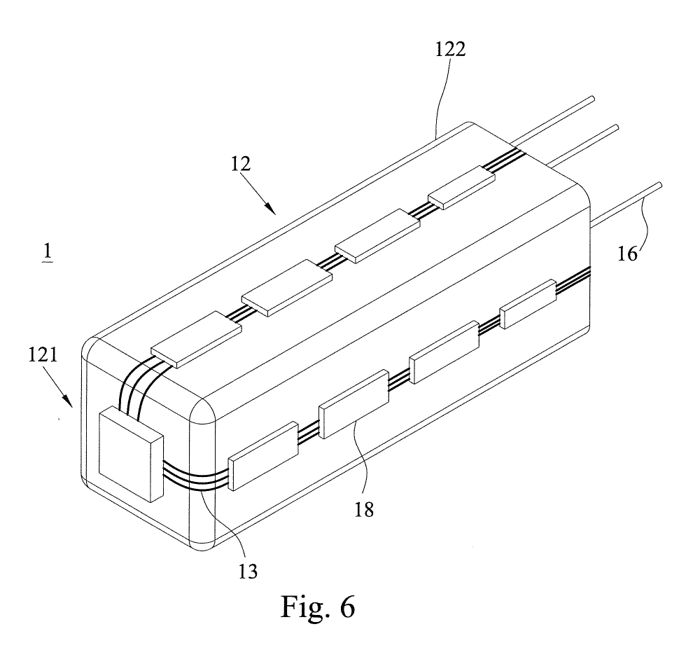

With reference to FIGS. 4 to 6 for schematic views of a 3D structure of a micro sensing apparatus in accordance with the third to fifth preferred embodiments of the present invention, the functions and components of the micro sensing apparatus of these preferred embodiments are substantially the same as those of the first preferred embodiment, and thus will not be described here again, and the only difference shown in the figure resides on that the connecting portion 121 and the carrying portion 122 of the micro sensing apparatus 1 are integrally formed, and the external diameter of the connecting portion 121 can be equal to or unequal to the external diameter of the carrying portion 122. In this preferred embodiment, the external diameter of the connecting portion 121 is equal to the external diameter of the carrying portion 122, and the cylindrical plastic body 12 is substantially in a circular cylindrical shape (as shown in FIG. 4), a triangular cylindrical shape (as shown in FIG. 5) or a rectangular cylindrical shape (as shown in FIG. 6), but the invention is not limited to these shapes only.

The micro sensing apparatus 1 further comprises a plurality of transmission lines 16 coupled to one of the circuits 13 or one of the electronic devices 18.

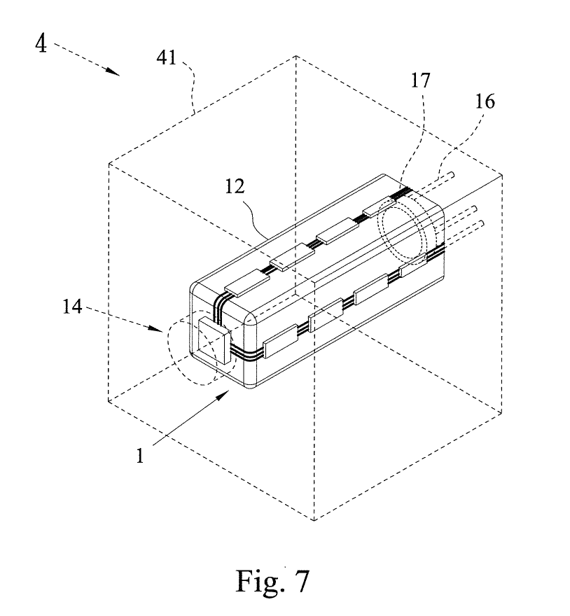

With reference to FIG. 7 for a schematic view of a 3D structure of a micro sensing apparatus in accordance with a sixth preferred embodiment of the present invention, the functions and components of the micro sensing apparatus of this preferred embodiment are substantially the same as those of the fifth preferred embodiment, and thus will not be described here again, and the only difference shown in the figure resides on that the interior of the cylindrical plastic body 12 is in a hollow form to facilitate accommodating or installing a plurality of transmission lines 16 or power storage devices 17 (such as batteries). The cylindrical plastic body 12 includes an external casing 41 for installing the micro sensing apparatus 1 therein, and a lens module 14 can be installed at a front end of the micro sensing apparatus 1 to become another device including not limited to a monitoring device 4.

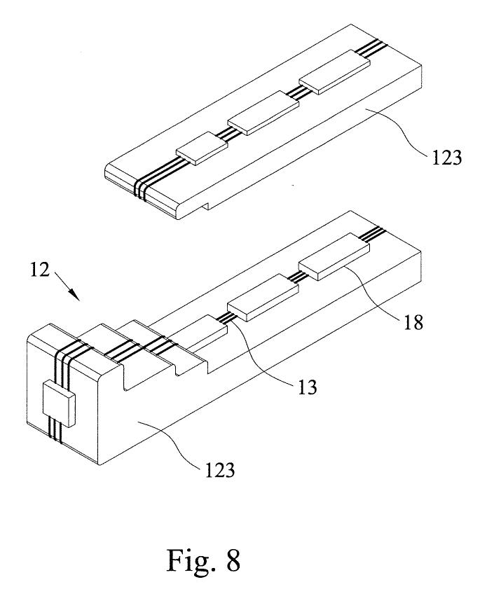

With reference to FIG. 8 for a schematic view of a 3D structure of a micro sensing apparatus in accordance with a seventh preferred embodiment of the present invention, the functions and components of the micro sensing apparatus of this preferred embodiment are substantially the same as those of the first preferred embodiment, and thus will not be described here again, and the only difference shown in the figure resides on that the cylindrical plastic body 12 is formed by combining a plurality of molding elements 123, and at least one side of each molding element 123 can be used for installing the electronic device 18. At least one side of each molding element 123 can be used for forming a plurality of circuits 13 and allowing the installation of more electronic devices 18.

The sensing device is installed to the cylindrical plastic body (which comprises integrally formed connecting portion and carrying portion) to overcome the problem of requiring the adapting mechanism for coupling the conventional sensing device with the printed circuit board to simplify the manufacturing procedure, improve the convenience, and overcome the drawback of soldering, adhering or snapping the adapting mechanism, so as to prevent the printed circuit board from falling out due to external vibration forces. With the circuits (including curved-surface circuits, three-dimensional circuits or a combination of curved-surface circuits and planar circuits) formed on the curve surface of the cylindrical plastic body, the image sensor and the electronic device can be connected easily for the transmission of electric signals.

In summation of the description above, the present invention improves over the prior art and complies with the patent application requirements, and thus is duly file for patent application. While the invention has been described by means of specific embodiments, numerous modifications and variations could be made thereto by those skilled in the art without departing from the scope and spirit of the invention set forth in the claims.

What is claimed is:

1. A micro sensing apparatus, comprising:
a sensing device, having a bottom surface;
a cylindrical plastic body, axially coupled to the sensing device, and further comprising:
a connecting portion, having a first end surface coupled to the bottom surface; and
a carrying portion, integrally formed and coupled to the connecting portion, and having a plurality of electronic devices installed thereon; and
a plurality of circuits, formed on the carrying portion.

2. The micro sensing apparatus of claim 1, wherein the micro sensing apparatus is manufactured by a molded interconnect device (MID) process or a laser direct structuring (LDS) process.

3. The micro sensing apparatus of claim 1, wherein the cylindrical plastic body is formed by an injection molding method, a hollow air-blowing molding method, a vacuum molding method or a hot-press molding method.

4. The micro sensing apparatus of claim 1, further comprising a lens module installed at a front end of the micro sensing apparatus to form an endoscopic device, and the lens module is aligned towards the sensing device.

5. The micro sensing apparatus of claim 4, further comprising a hollow pipe installed at an end of the micro sensing apparatus to become an endoscope, and the hollow pipe is disposed in proximity to the carrying portion.

6. The micro sensing apparatus of claim 1, wherein the cylindrical plastic body is in a circular cylindrical shape, a rectangular cylindrical shape , a triangular cylindrical shape, an elliptical cylindrical shape, a semi-circular cylindrical shape or a hyperbolic cylindrical shape.

7. The micro sensing apparatus of claim 1, wherein the carrying portion is substantially board-shaped or pillar-shaped.

8. The micro sensing apparatus of claim 1, wherein the connecting portion is substantially board-shaped or pillar-shaped.

9. The micro sensing apparatus of claim 1, wherein one of the circuits is a curved-surface circuit, a three-dimensional circuit, or a combination of the curved-surface circuit and a planar circuit.

10. The micro sensing apparatus of claim 1, wherein the sensing device is a complementary metal oxide semiconductor (CMOS) sensing device, a charge coupled device (CCD), a thermal sensing device, a pressure sensing device, a flow sensing device, a laser sensing device, a displacement sensing device, an ultrasonic sensing device or an infrared sensing device.
